(19) United States
(12) Patent Application Publication
(10) Pub. No.: US 2009/0011491 A1
(43) Pub. Date: Jan. 8, 2009

(54) Culture Media for Increasing Biopesticide Producing Microorganism's Pesticidal Activity, Methods of Producing Same, Biopesticide Producing Microorganisms so Produced

(76) Inventors: Simon Barnabe, Sainte-Foy (CA); Mausam Verma, Sainte-Foy (CA); Rajeshwar Dayal Tyagi, Sainte-Foy (CA); Jose R. Valero, Sainte-Foy (CA)

Correspondence Address: Kevin M. Farrell;Pierce Atwood LLP, One New Hampshire Ave., Suite 350, Portsmouth, NH, 03801, US

(21) Appl. No.: 11/884,850

(22) PCT Filed: Feb. 22, 2005
(86) PCT No.: PCT/CA2005/000235

§ 371 Date: Sep. 12, 2008

(51) Int. Cl. C12N 1/20; C12N 1/14

(52) U.S. Cl. 435/252.5; 435/253.6; 435/256.8; 435/256.7

(57) ABSTRACT

A media for growing a biopesticide producing microorganism comprising waste water sludge having undergone thermal alkaline hydrolysis performed by adjusting the pH of the wastewater sludge between about 8 and about 12 with an alkaline solution selected from the group consisting of NaOH, KOH, CaOH₂ and MgOH₂ at a temperature between about 120 and about 180 degree Celsius, methods using this media and biopestide producing microorganism so produced.

### FIELD OF THE INVENTION

The present invention relates to culture media for increasing biopesticide producing microorganisms' pesticidal activity, methods of producing same, and biopesticide producing microorganisms so produced. More specifically, the present invention relates to waste water sludges treated to increase the bioavailability of their components (in terms of solubility, concentration, metabolic conformity, decreasing in complexity or biodegradability for instance) and methods of using these sludges for growing microorganisms such as Bacillus thuringiensis and Trichoderma spp., or a recombinant microorganism capable of expressing a gene derived from a biopesticide producing microorganism encoding an entomotoxin and for increasing the pesticidal activity of these microorganims.

### BACKGROUND OF THE INVENTION

Pests pose a serious constraint to agricultural production, the losses estimated average almost 12% of the world's agricultural output alone (Jutsum, 1988). Synthetic chemical pesticides have long been used as active agents in mitigating diseases and other problems caused by insects, weeds, rodents, nematodes, fungi or pathogenic microorganisms (bacteria and virus). But their adverse impacts viz. extensive pollution and pathogen resistance induced a new era of biological control.

### Bacteria

Biopesticides producing bacteria exist that can be grown in alternative media. Based on Copping & Menn (2000) literature review, biopesticides producing bacteria are the following: Bacillus thuringiensis (‘BT’), Bacillus sphaericus, Bacillus subtilis, Agrobacterium radiobacter, Bulkholderia cepacia, Pseudomonas fluorencens, Pseudomonas syringae, Streptomyces griseoviridis. Works on growth of Bacillus sphaericus and Bacillus subtilis in pre-treated (or physico-chemically transformed) alternative media such as food industry by-products have been published.

As formulated and registered for more than 50 years, spore-forming BT is the most common bacteria used in the worldwide pesticide market.

BT is a motile, rod-shaped, gram-positive bacterium that is widely distributed in nature. During sporulation, BT produces a parasporal crystal inclusion(s) which is insecticidal upon ingestion to susceptible insect larvae of the order Lepidoptera, Diptera, or Coleoptera. The inclusion(s) may vary in shape, number, and composition. They are comprised of one or more proteins called crystal delta-endotoxins. The insecticidal crystal delta-endotoxins are generally converted by proteases in the larval gut into smaller (truncated) toxic polypeptides, causing cells midgut destruction, and ultimately, death of the insect. Other BT substance may have pesticidal activity, by synergism with insecticidal crystal or not. It includes spores, vegetative insecticidal protein, proteases, chitinases, lecithinases, hemeolysins, exotoxins (β, α, γ, σ) and other unknown proteins. There are several BT strains that are widely used as biopesticides in the forestry, agricultural, and public health areas. BT subspecie kurstaki and BT subspecie aizawai have been found to be specific against Lepidoptera. BT subspecie israelensis has been found to be specific for Diptera. Bacillus thuringiensis biovar tenebrionis (related to serovar morrisoni, BT tenebrionis is also called san diego) and BT serovar japonensis has been found to be specific for Coleoptera. Other entomopathogen strains of BT also have reported pesticidal activity against other insect orders (Hymenoptera, Homoptera, Orthoptera, Mallophaga), nematodes, mites and protozoa (Schnepf et al., 1998).

Cost-effective BT based and other microorganisms based biopesticides must still be developed to be more competitive against chemicals. According to Lisansky et al. (1993), the synthetic media normally used for BT production is costly for mass production: it may correspond to between 44 and 92% of the total production cost. Use of cheap alternative media has been proposed to increase the cost-effectiveness of BT based biopesticides. Tirado-Montiel et al. (1998) have published a review on several agricultural and industrial raw materials, products or by-products studied as alternative media for BT production. Alternative media are inexpensive substrates that support well BT growth, sporulation and insecticidal crystal production. Wastewater sludge for instance has been proposed as alternative media for BT production. Generally however, entomotoxicities of BT based biopesticides produced in cheap alternative media including wastewater sludge are equal to or less than entomotoxicities obtained using conventional synthetic media. In wastewater sludge for instance, most of the nutrients are unavailable, which prevents BT from achieving higher insecticidal activity (or entomotoxicity) values by producing more spores, insecticidal crystals or other insecticidal metabolites (e.g. vegetative insecticidal proteins) and metabolites contributing to entomotoxicity (e.g. chitinases).

Various methods for increasing nutrient bioavailability (in terms of concentration) in alternative media have been proposed to achieve higher entomotoxicity values. Tirado-Montiel (1997) has suggested to add glucose or yeast extract in wastewater sludge used as raw material for BT production to improve nutrient content of the sludge and increase BT yield (in terms of cells, spores and entomotoxicity). It was shown that addition of nutrient was however not enough to achieve an equivalent or a better entomotoxicity than standard soy based medium. Furthermore, addition of exogenous nutrient supplements is expensive.

Waste water sludges are complex materials. Components of interest for specific microbial production such as BT may be unavailable for bacteria metabolism (complex and hard to degrade, inadequate conformation for enzymatic activities, insoluble, lack of nutrients). In this context, attempts were made to modify waste water sludge for improving BT production (Tirado, 1997; Tirado-Montiel et al., 2001). Hence, Tirado-Montiel (1997 & 2001) have tested acid hydrolysis of wastewater sludge by which they improved entomotoxicity of BT produced in sludge by 24%. However, it was shown that acid hydrolysis did not improve entomotoxicity as compared to that obtained with standard soy based medium. Tirado-Montiel (1997 & 2001) achieved less than 4.1×10³ international units by liter (IU/μL) with this method, not much higher than the 3.8×10³ IU/μL obtained in standard soy based medium. Furthermore, it was shown that acid hydrolysis may destroy nutrients that are assimilated by BT. The present applicant have tested Tirado-Montiel (1997 & 2001)'s conditions to grow BT on sludges adjusted to 25 grams of suspended solids by liter (g SS/I). Not entomotoxicity increase was observed as compared to untreated sludge.

Ben Rebah et al. (2001) applied acid and alkaline hydrolysis to improve a Rhizobia bacteria, namely Sinorhizobium meliloti, cell production in waste water sludge. This bacteria is characterized by its ability to nodulate plant roots does not produce delta-endotoxin or spores. In this case, acid (pH 2) and alkaline (100 meq NaOH/L) pre-treatments increased cell count of S. meliloti by 10-fold and 2-fold respectively. This treatment did not seek to control pH.

A media's ability to increase bacteria cell growth is not correlated with its ability to increase the bacteria's entomotoxicity (i.e. spores & insecticidal secondary metabolites such as insecticidal crystal, vegetative insecticidal protein, proteases, chitinases and sometime exotoxines or other unknown proteins play a role in BT entomotoxicity, but not cell concentration). In fact, mechanisms for spores & insecticidal secondary metabolites are often repressed by those for cell growth. For instance, sporulation and insecticidal metabolites formation is inhibited through mechanisms such as catabolic repression by simple carbon sources (e.g. glucose) or nitrogen sources e.g. ammonia). Sludges treated according to Rebah's method did not increase BT's entomotoxicity.

Lachhab et al. (2001) showed that raw sludge fermentation by BT kurstaki HD-1 yielded low entomotoxicity (about 8×10³ IU/μl) when the SS was less than log/l. They thus proposed to increase waste water sludge solids concentration in order to improve nutrient content of wastewater sludge used as raw material for BT production. They however achieved entomotoxicity values of less than 9.8×10³ IU/μL at a solid concentration of 36 grams of solids by liter of sludge (g/L), an entomotoxicity value lower than that obtained at 26 g/L namely 13.0×10³ IU/μL. Lacchab thus showed that untreated/raw waste water sludge fermentation was optimal for entomotoxicity at 26 g/l. The use of solids in concentration beyond 26 g/L of sludge in and of itself hence did not increase the entomotoxicity value in spite of a potential increase in the nutrients (in terms of concentration). It is believed that a solid concentration higher that 26 g/L may affect oxygen transfer, which becomes a limiting factor for BT growth as well as spore and insecticidal metabolite production (Avignone-Rossa and Mignone, 1993). It is believed also that a solid concentration higher that 26 g/L may provoke substrate inhibition. Sludge particles and extracellular polymers may interfere with enzymatic activities or nutrient transport through cell membrane systems involved in spores and insecticidal crystal production (Vidyarthi et al., 2002).

### Fungus

Amongst biocontrol agents (BCAs), parasitic fungi penetrate directly their targets and are resistant to adverse environmental conditions. Trichoderma spp. are good examples of antagonistic fungi that have broader host specificity (insecticide and herbicide) and act simultaneous as a biofertiliser to favor plant growth (Babu et al., 2003), and are therefore good BCAs. Trichoderma spp. are facultative anaerobics, saprophytic parasitic fungi, which produce abundant conidia (spores) under specific environmental conditions and a wide range of enzymes-cellulases, proteases, chitinases, lipases and several antibiotics (Ortiz and Orduz, 2000).

A maximum of 33 taxa have been reported so far for this genus (Samuels et al. 2004). However, Trichoderma viride, Trichoderma ressei, Trichoderma harzianum, Trichoderma virens (earlier also known as Gliocladium virens), Trichoderma koningii, Trichoderma longibrachiatum and Trichoderma pseudokoningii are some common species of the genus which are considered to be very important as biopesticide producing species (Ejechia, 1997; Papavizas, 1985). Further, the significance of these species as biopesticide producers could be assessed from Table 1 below.

TABLE 1
List of Trichoderma spp. used as biocontrol agents

| Microorganism | Trade Name | Pests Controlled |
|---|---|---|
| Gliocladium spp.# | GlioMix ™ | Soil pathogens |
| Gliocladium virens# | Soil Guard 12G ™ | Soil pathogens that cause damping off |
| | | and root rot, esp. Rhizoctonia solani & |
| | | Pythium spp. |
| Trichoderma | RootShield ™ BioTrek | Soil pathogens - Pythium, Rhicozoktonia, |
| harzianum | 22G ™ Supresivit ™ | Verticillium, Sclerotium, and others |
| | T-22G ™, T-22HB ™ | |
| T. harzianum | Trichodex ™ | Botritis cinerea and others |
| T. harzianum | Binab ™ | Tree-wound pathogens |
| And T. polysporum | | |
| T. harzianum | Trichopel ™ | Armillaria, Botryoshaeria, and others |
| And T. viride | Trichojet ™ | |
| | Trichodowels ™ | |
| | Trichoseal ™ | |
| Trichoderma spp. | Promot ™ | Growth promoter, Rhizoctonia solani, |
| | Trichoderma 2000 | Sclerotium rolfsii, Pythium spp., Fusarium |
| | Biofungus | spp. on nursery and field crops |
| T. viride | Trieco | For management of Rhizoctonia spp., |
| | | Pythium spp., Fusarium spp., root rot, |
| | | seedling rot, collar rot, red rot, damping- |
| | | off, Fusarium wilt on wide variety of crops |

#The genus Gliocladium have been reclassified and included in the more rapidly expanding genus Trichoderma (Harmann and Björjmann, 1998).

Trichoderma spp. are potentially non-pathogenic fungi and therefore falls in the class of GRAS-listed (Generally Referred As Safe) microorganisms (Headon and Walsh, 1994). Also, many studies support the non-pathogenic nature of Trichoderma spp. (Benhamou and Brodeur, 2000; Benhamou et al., 1999; Chet, 1993). Furthermore, various species of this genus have been successfully used in the production of cellulolytic and hemicellulolytic enzymes of industrial importance, biological control of plant disease, biodegradation of chlorophenolic compounds, and soil bioremediation (Esposito and Manuela da Silva, 1998; Felse and Panda, 2000; Lisboa De Marco et al., 2003).

Several substrates have been explored for the production of Trichoderma spp. Conventionally raw material like, glucose, glucose nitrate, sucrose, molasses etc are used for Trichoderma viride production at laboratory and commercial levels. Several alternative substrates have been explored for the production of Trichoderma spp., either by solid state or submerged fermentation process, for example, vegetable oils, nutrient fortified peat moss, composted chicken manure, potato dextrose agar, corn cobs, wheat bran, cocoa shell meal, pine sawdust, peanut hull meal, sugar beet bagasse, corn stover, wheat straw, cornmeal and agricultural by-products (Feng et al., 1994; Steinmetz and Schonbeck, 1994; Bonnarme et al., 1997; Jones et al., 1988 Hutchinson, 1999; Howard et al., 2003). These raw materials proven to be non-economical either because of cost factor related to high demand (which results in high cost for some alternative raw materials), low yield in terms of product (conidia) formation (in all cases), longer fermentation time (ranging from 4-10 days in solid substrate production) and/or formulation cost (in all cases) (Felse and Panda, 2000). Other treatments require mandatory pre-treatment step(s) to achieve competitive production efficacy and possess some inherent problems such as being labour intensive, having scale-up constraints and poor process control.

### Sludge Management

Sludges have been posing serious problems of treatment and disposal, hence ecologically benign sustainable alternatives have been proposed to overcome the same. Bioconversion into value added products like biopesticides is one of the profitable and holistic approaches to mitigate the proliferating menace (Tirado-Montiel et al., 2001; Vidyarthi et al., 2002).

There remains a need for improved methods to increase nutrient availability in culture media for biopesticide producing microorganisms and a need for an improved culture media to increase biopesticide's pesticidal activity.

There also remains a need for improved biopesticide producing microorganisms for mass production.

There also remains a need for new sludge management methods.

### SUMMARY OF THE INVENTION

It is an object of the present invention to present methods to provide an improved culture media for increasing the pesticidal activity of biopesticide producing microorganims. It is also an object of the present invention to provide so produced medias and more effective biopesticide microorganisms.

More particularly, there is provided a media for growing a biopesticide producing microorganism comprising waste water sludge having undergone thermal alkaline hydrolysis performed by adjusting the pH of the wastewater sludge between about 8 and about 12 with an alkaline solution selected from the group consisting of NaOH, KOH, CaOH₂ and MgOH₂ at a temperature between about 120 and about 180 degree Celsius. In a more specific embodiment, the thermal alkaline hydrolysis is performed for at least about 10 minutes to about 50 minutes. In an other more specific embodiment, the sludge was oxidized after the heating step. In an other more specific embodiment, step of oxidizing the sludge was performed by adjusting the pH with a sulfuric acid solution at about 1.5 to about 4 and adding 3.19E-07 to 9.58E-07 kg H₂O₂ per gram of SS. In an other more specific embodiment, the sludge was after the oxidation step further placed in a heating bath up to 70 degree Celsius for about 1.5 to 4 hours. In an other more specific embodiment, the sludge has been subjected, after thermal alkaline hydrolysis, to a step of adjusting the sludge's pH with an acid which does not have an inhibitory effect on BT growth. In an other more specific embodiment, the acid is H₂SO₄. In an other more specific embodiment, the sludge has a concentration in solids between about 10 g SS/L and about 50 g SS/L. In an other more specific embodiment, the biopesticide producing microorganism is a biopesticide producing bacteria. In an other more specific embodiment, the biopesticide producing microorganism is a biopesticide producing Bacillus thuringiensis (BT). In an other more specific embodiment, the biopesticide producing BT is selected from the group consisting of BT serovar israelensis; BT biovar tenebrionis; BT serovar japonensis; and BT serovar aizawai. In an other more specific embodiment, the biopesticide producing microorganism is a biopesticide producing fungus. In an other more specific embodiment, the biopesticide producing microorganism is a biopesticide producing Trichoderma spp.

In accordance with the present invention, there is also provided a method for increasing the bioavailability of nutrients in waste water sludge for biopesticide producing microorganisms, comprising subjecting the sludge to a thermal alkaline pre-treatment comprising adjusting the sludge pH to between about 8 and about 12 at a temperature between about 120 and 180 degree Celsius for a time sufficient to increase the bioavailability of nutrients in said sludge.

In accordance with the present invention, there is also provided a method of increasing the pesticidal activity of a biopesticide producing microorganism, comprising growing a biopesticide producing microorganism in a culture media of the present invention. In an other more specific embodiment, the biopesticide producing microorganism is a biopesticide producing bacteria. In an other more specific embodiment, the biopesticide producing microorganism is a biopesticide producing Bacillus thuringiensis (BT). In an other more specific embodiment, the biopesticide producing BT is selected from the group consisting of BT serovar israelensis; BT biovar tenebrionis; BT serovar japonensis; and BT serovar aizawai. In an other more specific embodiment, the biopesticide producing microorganism is a biopesticide producing fungus. In an other more specific embodiment, the biopesticide producing microorganism is a biopesticide producing Trichoderma spp.

In accordance with the present invention, there is also provided a method of increasing the pesticidal activity of a biopesticide producing microorganism, comprising (a) subjecting waste water sludge to a thermal alkaline pre-treatment comprising adjusting the sludge pH to between about 8 and between about 12 at a temperature between about 120 and 180 degree Celsius for a time sufficient to increase the bioavailability of nutrients in said sludge; (b) adjusting the pH of the sludge to provide appropriate growth conditions for the biopesticide producing microorganism; and (c) growing the biopesticide producing microorganism in the sludge of step (b). In an other more specific embodiment, the thermal alkaline hydrolysis is performed for at least about 10 minutes. In an other more specific embodiment, the method further comprises the step of oxidizing the sludge after step (a). In an other more specific embodiment, the step of oxidizing the sludge is performed by adjusting the pH with a sulfuric acid solution at about 1.5 to about 4 and adding 3.19E-07 to 9.58E-07 kg of H₂O₂ per gram of SS. In an other more specific embodiment, the method further comprises after the oxidation step, the step of placing the sludge in a heating bath at about 25 to 70 degree Celsius for about 1.5 to 4 hours. In an other more specific embodiment, the said sludge has a concentration in solids between about 10 g SS/L and about 50 g SS/L prior to step (a). In an other more specific embodiment, the biopesticide producing microorganism is a biopesticide producing bacteria. In an other more specific embodiment, the biopesticide producing microorganism is a biopesticide producing Bacillus thuringiensis (BT). In an other more specific embodiment, the biopesticide producing BT is selected from the group consisting of BT serovar israelensis; BT biovar tenebrionis; BT serovar japonensis; and BT serovar aizawai. In an other more specific embodiment where the biopesticide producing microorganism is a biopesticide producing bacteria, the pH to which the sludge is adjusted at step (b) is 7.0±0.2. In an other more specific embodiment, the biopesticide producing microorganism is a biopesticide producing fungus. In an other more specific embodiment, the biopesticide producing microorganism is a biopesticide producing Trichoderma spp. In an other more specific embodiment where the biopesticide producing microorganism is a biopesticide producing fungus, the pH to which the sludge is adjusted at step (b) is 6.1±0.1. In an other more specific embodiment, the pH is adjusted at step (b) with H₂SO₄.

In accordance with the present invention, there is also provided a biologically pure biopesticide producing microorganism grown in a culture media of the present invention.

In accordance with the present invention, there is also provided a biologically pure biopesticide producing microorganism produced by a method of the present invention.

As used herein the term “BT” is meant to encompass any strain of BT including novel strains that could be isolated from wastewater sludges. These strains are adapted to their environment and are very efficient when grown in wastewater sludges when using prior art microbial culture methods (i.e. sterilizing culture media prior to growing the bacteria). Without limiting the foregoing, it includes the following BT:

B. THURINGIENSIS STRAINS BY SUBSPECIES

| Serovar | Serotype | BGSC No. |
|---|---|---|
| aizawai/pacificus | 7 | 4J1-4J5 |
| alesti | 3a, 3c | 4C1-4C3 |
| amagiensis | 29 | 4AE1 |
| andalousiensis | 37 | 4AW1 |
| argentinensis | 58 | 4BV1 |
| asturiensis | 53 | 4BQ1 |
| azorensis | 64 | 4CB1 |
| balearica | 48 | 4BK1 |
| brasilensis | 39 | 4AY1 |
| cameroun | 32 | 4AF1 |
| canadensis | 5a, 5c | 4H1-4H2 |
| chanpaisis | 46 | 4BH1 |
| colmeri | 21 | 4X1 |
| coreanensis | 25 | 4AL1 |
| dakota | 15 | 4R1 |
| darmstadiensis | 10a, 10b | 4M1-4M3 |
| entomocidus/subtoxicus | 6 | 4I1-4I5 |
| finitimus | 2 | 4B1-4B2 |
| fukuokaensis | 3a, 3d, 3e | 4AP1 |
| galleriae | 5a, 5b | 4G1-4G6 |
| graciosensis | 66 | 4CD1 |
| guiyangiensis | 43 | 4BC1 |
| higo | 44 | 4AU1 |
| huazhongensis | 40 | 4BD1 |
| iberica | 59 | 4BW1 |
| indiana | 16 | 4S2-4S3 |
| israelensis | 14 | 4Q1-4Q8 |
| japonensis | 23 | 4AT1 |
| jegathesan | 28a, 28C | 4CF1 |
| jinghongiensis | 42 | 4AR1 |
| kenyae | 4a, 4c | 4F1-4F4 |
| kim | 52 | 4BP1 |
| konkukian | 34 | 4AH1 |
| kumamtoensis | 18a, 18b | 4W1 |
| kurstaki | 3a, 3b, 3c | 4D1-4D21 |
| kyushuensis | 11a, 11c | 4U1 |
| leesis | 33 | 4AK1 |
| londrina | 10a, 10c | 4BF1 |
| malayensis | 36 | 4AV1 |
| mexicanensis | 27 | 4AC1 |
| monterrey | 28a, 28b | 4AJ1 |
| morrisoni | 8a, 8b | 4K1-4K3 |
| muju | 49 | 4BL1 |
| navarrensis | 50 | 4BM1 |
| neoleonensis | 24a, 24b | 4BE1 |
| nigeriensis | 8b, 8d | 4AZ1 |
| novosibirsk | 24a, 24c | 4AX1 |
| ostriniae | 8a, 8c | 4Z1 |
| oswaldocruzi | 38 | 4AS1 |
| pakistani | 13 | 4P1 |
| palmanyolensis | 55 | 4BS1 |
| pingluonsis | 60 | 4BX1 |
| pirenaica | 57 | 4BU1 |
| poloniensis | 54 | 4BR1 |
| pondicheriensis | 20a, 20c | 4BA1 |
| pulsiensis | 65 | 4CC1 |
| rongseni | 56 | 4BT1 |
| roskildiensis | 45 | 4BG1 |
| seoulensis | 35 | 4AQ1 |
| shanongiensis | 22 | 4AN1 |
| silo | 26 | 4AG1 |
| sooncheon | 41 | 4BB1 |
| sotto/dendrolimus | 4a, 4b | 4E1-4E4 |
| sumiyoshiensis | 3a, 3d | 4AO1 |
| sylvestriensis | 61 | 4BY1 |
| thompsoni | 12 | 4O1 |
| thuringiensis | 1 | 4A1-4A9 |
| tochigiensis | 19 | 4Y1 |
| toguchini | 31 | 4AD1 |
| tohokuensis | 17 | 4V1 |
| tolworthi | 9 | 4L1-4L3 |
| toumanoffi | 11a, 11b | 4N1 |
| vazensis | 67 | 4CE1 |
| wratislaviensis | 47 | 4BJ1 |
| wuhanensis | none | 4T1 |
| xiaguangiensis | 51 | 4BN1 |
| yunnanensis | 20a, 20b | 4AM1 |
| zhaodongensis | 62 | 4BZ1 |

In preferred embodiment and without limiting the generality of the foregoing, this term refers to entomopathogenic BT. This includes BT serovar israelensis; BT biovar tenebrionis; BT biovar san diego; BT serovar japonensis; and BT serovar aizawai.

As used herein the term “biopesticide” refers to a microorganism derived material or compound, or a combination of same, possessing pesticidal activity (amount of activity against a pest through killing, stunting of the growth, provoking sub-lethal effects or sickness, or protecting against pest infestation). Without being so limited, it includes any entomotoxic material or compound or combination of same produced by Bacillus thuringiensis (“BT”), Bacillus sphaericus, Bacillus subtilis, Agrobacterium radiobacter, Bulkholderia cepacia, Pseudomonas fluorencens, Pseudomonas syringae, Streptomyces griseoviridis, Trichoderma viride, Trichoderma virens, Trichoderma harzianum, Verticillium lecanii, Beauveria bassiana, Colletotrichum gloeosporioides. With regards to BT, the term biopesticide also includes other BT substance or mixture of substances that may have pesticidal activity, by synergism with insecticidal crystal or not. It includes entomotoxic microorganism derived spores, vegetative insecticidal protein, proteases, chitinases, lecithinases, hemeolysins, exotoxins (β, α, γ, σ) and any fragment thereof and other unknown proteins and combination thereof. In Examples presented herein, the biopesticides material or compounds disclosed include Trichoderma spp. conidia and BT produced crystal delta-endotoxins and spores.

As used herein the terminology “BT entomotoxicity” refers to the pesticidal activity (amount of activity against a insect pest through killing, stunting of the growth, provoking sub-lethal effects or sickness, or protecting against insect pest infestation) expressed by a BT biopesticide or by a microorganism capable of expressing a BT gene encoding said BT protein or fragment thereof. Such microorganism, capable of expressing a BT gene encoding a BT biopesticide inhabits the phylloplane (the surface of the plant leaves), and/or the rhizosphere (the soil surrounding plant roots), and/or aquatic environments, and is capable of successfully competing in the particular environment (crop and other insect habitats) with the wild-type microorganisms and provide for the stable maintenance and expression of a BT gene encoding a BT protein or fragment thereof with activity against or which kill pests. Examples of such microorganisms include, but are not limited to, bacteria, e.g., genera Bacillus, Pseudomonas, Erwinia, Serratia, Klebsiella, Xanthomonas, Streptomyces, Rhizobium, Rhodopseudomonas, Methylophilius, Agrobacterium, Acetobacter, Lactobacillus, Arthrobacter, Azotobacter, Leuconostoc, Alcaligenes, and Clostridium; algae, e.g., families Cyanophyceae, Prochlorophyceae, Rhodophyceae, Dinophyceae, Chrysophyceae, Prymnesiophyceae, Xanthophyceae, Raphidophyceae, Bacillariophyceae, Eustigmatophyceae, Cryptophyceae, Euglenophyceae, Prasinophyceae, and Chlorophyceae; and fungi, particularly yeast, e.g., genera Saccharomyces, Cryptococcus, Kluyveromyces, Sporobolomyces, Rhodotorula, and Aureobasidium. Pests may be an insect, a nematode, a mite, a protozoa or a snail.

A recombinant microorganism expressing BT genes is obtained by standard procedures for isolating plasmid DNA, cloning experiments and other DNA manipulations were as described by Sambrook et al. (1989). For the invention, they are given only by way of example and are not intended to limit the scope of the claims herein: transfer of cloned delta-endotoxin genes, or a DNA segment encoding a crystal protein, into Bacillus thuringiensis, as well as into other organisms, may be achieved by a variety of techniques, including, but not limited to, protoplasting of cells; electroporation; particle bombardment; silicon carbide fiber-mediated transformation of cells; conjugation; or transduction by bacteriophage. As used herein, the term “DNA segment” refers to a DNA molecule that has been isolated free of total genomic DNA of a particular species. Therefore, a DNA segment encoding a crystal protein or peptide refers to a DNA segment that contains crystal protein coding sequences yet is isolated away from, or purified free from, total genomic DNA of the species from which the DNA segment is obtained, which in the instant case is the genome of the Gram-positive bacterial genus, Bacillus, and in particular, the species known as BT. Included within the term “DNA segment”, are DNA segments and smaller fragments of such segments, and also recombinant vectors, including, for example, plasmids, cosmids, phagemids, phage, viruses, and the like. The invention may also implies a mutant BT strain which produces a larger amount of and/or larger crystals than the parental strain. A “parental strain” as defined herein is the original BT strain before mutagenesis. To obtain such mutants, the parental strain may, for example, be treated with a mutagen by chemical means such as N-methyl-N′-nitro-N-nitrosoguanidine or ethyl methanesulfonate, or by irradiation with gamma rays, X-rays or UV. Specifically, in one method of mutating BT and selecting such mutants the following procedure is used: i) the parental strain is treated with a mutagen; ii) the thus presumptive mutants are grown in a medium suitable for the selection of a mutant strain; and iii) the mutant strain is selected for increased production of delta-endotoxin. Alternatively, the mutant(s) may be obtained using recombinant DNA methods known in the art. For example, a DNA sequence containing a gene coding for a delta-endotoxin may be inserted into an appropriate expression vector and subsequently introduced into the parental strain using procedures known in the art. Alternatively, a DNA sequence containing a gene coding for a delta-endotoxin may be inserted into an appropriate vector for recombination into the genome and subsequent amplification (Sambrook, J., E. F. Fritsch & T. Maniatis. (1989). Molecular Cloning: A Laboratory Manual, 2nd ed., Cold Spring Harbor Laboratory.).

In the past, genetic nomenclature organization of cry genes were relied on the insecticidal actitivities of the crystal protein against specific insect order (lepidoptera, diptera, coleoptera). Revision of nomenclature has been achieved since the discovery of new cry genes that were highly similar to known genes, but did not encode for a toxin with a similar insecticidal spectrum. Thus, a new nomenclature was developed which systematically classifies the Cry proteins based upon amino acid sequence homology rather than upon insect target specificities. This classification scheme, including most of the known toxins, is summarized in Table 1 below. Adapted from: Crickmore, N. & al. (1998). Microbiol. Mol. Biol. Rev., 62: 807-813. Any of these genes may be used in recombinant micro-organisms according to the present invention.

TABLE 1
KNOWN B. THURINGIENSIS delta-ENDOTOXINS, GENBANK
ACCESSION NUMBERS, AND REVISED NOMENCLATURE

| | | | | | GenBank |
|---|---|---|---|---|---|
| | | GenBank | | | Acces- |
| New | Old | Accession# | New | Old | sion# |
| Cry1Aa1 | CryIA(a) | M11250 | Cry1Eb1 | CryIE(b) | M73253 |
| Cry1Aa2 | CryIA(a) | M10917 | Cry1Fa1 | CryIF | M63897 |
| Cry1Aa3 | CryIA(a) | D00348 | Cry1Fa2 | CryIF | M63897 |
| Cry1Aa4 | CryIA(a) | X13535 | Cry1Fb1 | PrtD | Z22512 |
| Cry1Aa5 | CryIA(a) | D17518 | Cry1Ga1 | PrtA | Z22510 |
| Cry1Aa6 | CryIA(a) | U43605 | Cry1Ga2 | CryIM | Y09326 |
| Cry1Ab1 | CryIA(b) | M13898 | Cry1Gb1 | CryH2 | U70725 |
| Cry1Ab2 | CryIA(b) | M12661 | Cry1Ha1 | PrtC | Z22513 |
| Cry1Ab3 | CryIA(b) | M15271 | Cry1Hb1 | | U35780 |
| Cry1Ab4 | CryIA(b) | D00117 | Cry1Ia1 | CryV | X62821 |
| Cry1Ab5 | CryIA(b) | X04698 | Cry1Ia2 | CryV | M98544 |
| Cry1Ab6 | CryIA(b) | M37263 | Cry1Ia3 | CryV | L36338 |
| Cry1Ab7 | CryIA(b) | X13233 | Cry1Ia4 | CryV | L49391 |
| Cry1Ab8 | CryIA(b) | M16463 | Cry1Ia5 | CryV | Y08920 |
| Cry1Ab9 | CryIA(b) | X54939 | Cry1Ib1 | CryV | U07642 |
| Cry1Ab10 | CryIA(b) | A29125 | Cry1Ja1 | ET4 | L32019 |
| Cry1Ac1 | CryIA(c) | M11068 | Cry1Jb1 | ET1 | U31527 |
| Cry1Ac2 | CryIA(c) | M35524 | Cry1Ka1 | | U28801 |
| Cry1Ac3 | CryIA(c) | X54159 | Cry2Aa1 | CryIIA | M31738 |
| Cry1Ac4 | CryIA(c) | M73249 | Cry2Aa2 | CryIIA | M23723 |
| Cry1Ac5 | CryIA(c) | M73248 | Cry2Aa3 | | D86084 |
| Cry1Ac6 | CryIA(c) | U43606 | Cry2Ab1 | CryIIB | M23724 |
| Cry1Ac7 | CryIA(c) | U87793 | Cry2Ab2 | CryIIB | X55416 |
| Cry1Ac8 | CryIA(c) | U87397 | Cry2Ac1 | CryIIC | X57252 |
| Cry1Ac9 | CryIA(c) | U89872 | Cry3Aa1 | CryIIIA | M22472 |
| Cry1Ac10 | CryIA(c) | AJ002514 | Cry3Aa2 | CryIIIA | J02978 |
| Cry1Ad1 | CryIA(d) | M73250 | Cry3Aa3 | CryIIIA | Y00420 |
| Cry1Ae1 | CryIA(e) | M65252 | Cry3Aa4 | CryIIIA | M30503 |
| Cry1Ba1 | CryIB | X06711 | Cry3Aa5 | CryIIIA | M37207 |
| Cry1Ba2 | | X95704 | Cry3Aa6 | CryIIIA | U10985 |
| Cry1Bb1 | ET5 | L32020 | Cry3Ba1 | CryIIIB | X17123 |
| Cry1Bc1 | CryIb(c) | Z46442 | Cry3Ba2 | CryIIIB | A07234 |
| Cry1Bd1 | CryE1 | U70726 | Cry3Bb1 | CryIIIB2 | M89794 |
| Cry1Ca1 | CryIC | X07518 | Cry3Bb2 | CryIIIC(b) | U31633 |
| Cry1Ca2 | CryIC | X13620 | Cry3Ca1 | CryIIID | X59797 |
| Cry1Ca3 | CryIC | M73251 | Cry4Aa1 | CryIVA | Y00423 |
| Cry1Ca4 | CryIC | A27642 | Cry4Aa2 | CryIVA | D00248 |
| Cry1Ca5 | CryIC | X96682 | Cry4Ba1 | CryIVB | X07423 |
| Cry1Ca6 | CryIC | X96683 | Cry4Ba2 | CryIVB | X07082 |
| Cry1Ca7 | CryIC | X96684 | Cry4Ba3 | CryIVB | M20242 |
| Cry1Cb1 | CryIC(b) | M97880 | Cry4Ba4 | CryIVB | D00247 |
| Cry1Da1 | CryID | X54160 | Cry5Aa1 | CryVA(a) | L07025 |
| Cry1Db1 | PrtB | Z22511 | Cry5Ab1 | CryVA(b) | L07026 |
| Cry1Ea1 | CryIE | X53985 | Cry5Ba1 | PS86Q3 | U19725 |
| Cry1Ea2 | CryIE | X56144 | Cry6Aa1 | CryVIA | L07022 |
| Cry1Ea3 | CryIE | M73252 | Cry6Ba1 | CryVIB | L07024 |
| Cry1Ea4 | | U94323 | Cry7Aa1 | CryIIIC | M64478 |
| Cry7Ab1 | CryIIICb | U04367 | Cry18Aa1 | CryBP1 | X99049 |
| Cry8Aa1 | CryIIIE | U04364 | Cry19Aa1 | Jeg65 | Y08920 |
| Cry8Ba1 | CryIIIG | U04365 | Cry20Aa1 | | U82518 |
| Cry8Ca1 | CryIIIF | U04366 | Cry21Aa1 | | I32932 |
| Cry9Aa1 | CryIG | X58120 | Cry22Aa1 | | I34547 |
| Cry9Aa2 | CryIG | X58534 | Cyt1Aa1 | CytA | X03182 |
| Cry9Ba1 | CryIX | X75019 | Cyt1Aa2 | CytA | X04338 |
| Cry9Ca1 | CryIH | Z37527 | Cyt1Aa3 | CytA | Y00135 |
| Cry9Da1 | N141 | D85560 | Cyt1Aa4 | CytA | M35968 |
| Cry10Aa1 | CryIVC | M12662 | Cyt1Ab1 | CytM | X98793 |
| Cry11Aa1 | CryIVD | M31737 | Cyt1Ba1 | | U37196 |
| Cry11Aa2 | CryIVD | M22860 | Cyt2Aa1 | CytB | Z14147 |
| Cry11Ba1 | Jeg80 | X86902 | Cyt2Ba1 | “CytB” | U52043 |
| Cry12Aa1 | CryVB | L07027 | Cyt2Ba2 | “CytB” | AF020789 |
| Cry13Aa1 | CryVC | L07023 | Cyt2Ba3 | “CytB” | AF022884 |
| Cry14Aa1 | CryVD | U13955 | Cyt2Ba4 | “CytB” | AF022885 |
| Cry15Aa1 | 34 kDa | M76442 | Cyt2Ba5 | “CytB” | AF022886 |
| Cry16Aa1 | Cbm71 | X94146 | Cyt2Bb1 | | U82519 |
| Cry17Aa1 | Cbm71 | X99478 | | | |

As used herein, the terminology “biologically pure” strain is intended to mean a strain separated from materials with which it is normally associated in nature. Note that a strain associated with other strains, or with compounds or materials (e.g. waste water sludges) that it is not normally found with in nature, is still defined as “biologically pure.” A monoculture of a particular strain is, of course, “biologically pure.”

As used herein, the term “waste water sludge” refers to sludges containing mostly organic matters, namely municipal waste water sludge, industrial waste water sludge, swine manure or a combination of any of these sludges.

As used herein the terminology “municipal waste water sludge” refers to a sludge obtained from the treatment of spent or used (i.e. waste) water from urban or rural waste water treatment plants which receive waste water from sources such as combined sewer/separate storm overflows, households and commercial sanitaries and, sometimes, from industries. In these plants, waste water generally undergo primary treatment and sometimes secondary treatments that are of a physical, biological and/or chemical nature (EPA, 2004; GEMET, 2004) and that yield floating solids, deposits, sediments and viscous masses i.e. fractions more concentrated in solids than the inputted waste water. The municipal waste water sludge refers to any of all of these fractions. A person of ordinary skill in the art will understand that the content of municipal waste water sludge will vary depending on many factors including whether it contains wastes from industries, and if so, on what is the nature of the industries; on the types of treatments to which the waste water is subjected in the plant, etc. The methods of the present invention applies to sludges that are mostly organic in nature and thus contain the nutrients necessary for growing microorganism. These nutrients are better described herein below.

As used herein the terminology “industrial waste water sludge” refers to waste water sludges containing mostly organic matters resulting from industrial processes and manufacturing, namely secondary sludges from pulp & paper industries and sludges from the starch industry and from the potatoes transformation industries. These sludges have in common their high content in organics. These sludges are in practice either disposed of separately or combined with municipal sludge for final disposal.

As used herein the terminology “primary treatment” refers to the removal of floating solids and suspended solids, both fine and coarse, from municipal waste water (GEMET, 2004). As used herein the terminology “primary sludge” or “primary waste water sludge” refers to sludge generated by primary treatment.

As used herein the terminology “secondary treatment” refers a biological treatment in which biological organisms decompose most of the organic matter of the primary sludges into a innocuous, stable form (EPA, 2004; GEMET, 2004). As used herein the terminology “secondary sludges” refers to sludge generated by secondary waste water treatment. Current secondary treatments include the use of any of activated sludge processes, sequential batch reactors, biological discs, biofiltration, lagoons (aerated or not aerated) and anaerobic treatments. Of course, biological processes used to produce secondary sludges may change with time.

As used herein the term “pre-treatment” refers to the treatment to which primary, secondary, mixed or combined sludge is subjected to increase its bioavailability according to the present invention.

As used herein the terminology “mixed or combined sludges” refers to a mixture or combination of primary sludge and secondary sludge. The constituents of primary sludge and secondary sludge differ. Primary sludge and thus mixed sludge contains more organic matter than secondary sludge, which contain more living and dead microbial cells.

It is believed that the pesticidal activity of biopesticide producing microorganisms will always increase when grown in sludges treated according the methods of the present invention. However, the pesticidal activity so achieved may vary from one type of sludge to another. Indeed, the quality and quantity of proteins available in sludges may affect the pesticidal activity of biopesticide producing microorganisms that are grown in these sludges. There are a number of factors that are sources of variations for physico-chemical properties of sludges: 1) seasonal variations of waste water treatment plant affluent caused for instance by rain, snow melt, sewer flooding, salt from winter road treatment, fallen leaves in fall; 2) nature and content of industrial effluents discharged in sewers which may vary according to activities in these industries, (i.e. industrial charge of waste water treatment plant affluent): 3) type of primary and secondary waste water treatment as well as indoor or outdoor climatic conditions; 4) sludge retention time during sludge treatment or sludge age; 6) sludges manipulation conditions. Also, the methods of the present invention may dissolve proteins in secondary sludge. Proteins will thus become directly available to bacteria. The methods of the present invention will simplify protein in mixed sludge, but will not dissolve them. The bacteria will thus have to use its enzymes to further degrade protein so as to assimilate them. However, when the solid concentration of the sludges is constant, the pesticidal activity is expected to remain substantially constant.

Sludges treated according to the present invention should contain all elements required for microorganisms vegetative growth, sporulation and production of pesticidal factors. In most cases therefore, the sludges will contain an organic load comprising in suspended or dissolved form major elements (carbon in the form of polymers such as starch or monomers such as glucose, nitrogen contained in ammonium and polymers such as proteins or monomers as amino acids); and minor elements such as P, Ca, Mg, Mn, Cu, Zn, Na, K, Fe, Al and S. These minor elements are contained in organic molecules of living cells, cell fragments or extracellular matrix. The organic load also contains trace elements such as Cd, Cr, Mo, Ni, Pb, etc.; and growth factors such as vitamins and essential amino acids not synthesised by the microorganisms. The sludges organic load available to microorganisms will often be found mostly in the suspended matters in practicing the present invention. Indeed, waste water sludges is often transported to thickener and/or stored before it is used for the method of the present invention, and most of organic load initially present in dissolved form in the sludges is consumed during those storage and concentration steps.

A high sludge viscosity interferes with mass transfer (O₂ and nutrient) which limits the ability of the microorganisms to consume substrate, thereby, inhibiting production of pesticidal products. The methods of the present invention are able to decrease the sludge viscosity, hence helping increasing mass transfer and thus permit the use of a sludge concentration higher than those of the prior art.

As used herein the terminology “increasing the bioavailability of nutrients” refers to an increase of solubility, concentration, metabolic conformity and to an organic complexity decrease.

The present pre-treatment may successfully be applied on any type of waste water sludge: (i) primary sludge; (ii) secondary sludge; (iii) mixture or combination of primary and secondary sludges; (iv) biological sludges (different from secondary sludge, but generated by biological treatment of solid, semi-solid or liquid wastes); (v) thickened, stabilized (digested or decontaminated), and conditioned (dewatered or dry) sludges. Silica particles sometimes found in primary sludges are however desirably removed prior to treatment so that they do not interfere with fermentation equipment. In mixed sludges however, silica particles are in such low concentration that they generally do not interfere. The origin of the waste water sludge may be municipal, industrial or be raw swine manure.

As used herein the terminology “suspended solids” (SS) refers to solids particles suspended in water, which can be removed by filtration or settlement. Without being so limited SS can be measured in sludge as follows (according to APHA, 1989): (i) the sludges are centrifuged at 8000 (7650 g) revolution per minute during 15 minutes; (ii) the sludge pellet is dried at 105° C. during more than 1 hour to yield a dried pellet; (iii) the sludge supernatant is filtrated on a 1.5 mm pores filter, the filtered residue is then dried at 105° C. during more than 1 hour to yield a dried filtered residue; (iv) the dried pellet obtained at step ii) is weighed; (v) the dried filtered residue obtained at step iii) is weighed; (vi) SS calculation is made with initial sludge volume before centrifugation.

As defined herein, “IU” is meant to refer to international units as determined by bioassay. The bioassay compares the sample to standard Bacillus reference material using Trichoplusia ni or an other pest as the standard test insect (reference: Dulmage, H. T., O. P. Boening, C. S. Rehnborg& G. D. Hansen (1971). A proposed standardized bioassay for formulations of Bacillus thuringiensis based on the international unit. Journal of invertebrate pathology, 18: 240-245).

The alkaline hydrolysis of the present invention may be performed using bases such as NaOH, KOH, CaOH₂ and MgOH₂. NaOH however possesses the additional advantage of providing additional sodium to the sludges which was shown to further increase pesticidal activity of microorganisms that are grown in it.

Other objects, advantages and features of the present invention will become more apparent upon reading of the following non-restrictive description of preferred embodiments thereof, given by way of example only with reference to the accompanying drawings.

The present invention seeks to meet these needs and other needs.

The present description refers to a number of documents, the content of which is herein incorporated by reference in their entirety.

### BRIEF DESCRIPTION OF THE DRAWINGS

In the appended drawings:

FIG. 1 presents entomotoxicity values and spores concentrations of BT after 48 h in shake flask microbial culture with pre-treated waste water sludge (ta=thermal-alkaline hydrolysis, tao=thermal-alkaline hydrolysis following by partial oxidation) or raw wastewater sludge (none=no pre-treatment) and corresponding suspended solids content (ss/l). pre-treatments experiments shown are those in which the highest entomotoxicity values have been achieved;

FIG. 2 presents the CFU production profile of Trichoderma viride in raw sludge; and

FIG. 3 presents the CFU profile of Trichoderma viride in thermal alkaline treated sludge.

### DESCRIPTION OF ILLUSTRATIVE EMBODIMENTS

The invention proposes physico-chemical pre-treatments to partially solubilize waste water sludge and increase its potential to increase biopesticide producing microorganisms pesticidal activity. The present method allows the use of a higher sludge solid concentration while providing an increased nutrients bioavailability so as to achieve higher pesticidal activity values. The present invention concerns alkaline hydrolysis methods for partially solubilizing nutrients and other components in waste water sludge used as microbial culture substrate for biopesticide producing microorganisms production.

The present invention is illustrated in further details by the following non-limiting examples.

### EXAMPLE 1

### Origin of BT Strain Used

Bacillus thuringiensis var. kurstaki HD-1 (ATCC 33679) (Btk) was used. An active culture was maintained by streak inoculating tryptic soy agar™ (Difco), incubated at 30 degree Celsius for 48 hours and then stored at 4 degree Celsius for future use.

Procedure for Starter Culture and Acclimated Pre-Culture of BT

A loopful of BT colony from a tryptic soy agar plate was used to inoculate 100 ml of sterile tryptic soy broth (Difco) in 500 ml shake flask (Pyrex) to make the starter culture. Starter culture was incubated in a rotary shaker-incubator at 30 degree Celsius and 250 rounds per minute for 8 hours. To reduce lag phase of BT at the beginning of each experiment, a sludge inoculum (or acclimated pre-culture) was prepared by adding 2 ml of a starter culture into 100 ml of sterile waste water sludge placed in 500 ml shake flask. The sludge inoculum was incubated in a rotary shaker-incubator at 30 degree Celsius and 250 rounds per minute for 10 hours to 12 hours. Waste water sludge was sterilized at 121 degree Celsius during 30 minutes after adjusting pH to 7.0±0.2 with sulfuric acid solution or sodium hydroxide solution. Although a pH of 7.0±0.2 is believed to be optimal for growing most bacteria, it is expected that a pH of between about 6.6 and 7.4 will also be appropriate for culture. It has been shown however that at 6.5, microbial growth of BT is more limited. Growing BT in a sludge with a alkaline or acid pH at the beginning may cause a stress in the bacterial population, which may result in the lost of the plasmid that contain delta-endotoxin gene or in a premature beginning of the sporulation.

### BT Production

BT was produced by conventional microbial culture methods using waste water sludge as raw material. Pure microbial culture was conducted in 500 ml shake flasks (work volume of 100 mL). Bioreactors could be used instead of shake flasks for higher scale experiments, for example, 15 L and 150 μL stirred tank bioreactors (work volume of 10 L and 100 L respectively). BT production was conducted in batch culture. Fed-batch and continuous cultures can be conducted when bioreactor is used.

### Procedure for Shake Flask Experiment

Experiments were conducted in 500 ml shake flask containing 100 ml of sterile sludge (sterilized at 121 degree Celsius during 30 minutes after adjusting pH to 7.0±0.2 with sulfuric acid solution or sodium hydroxide solution.). Sludge was inoculated by adding 2 ml (2% v/v) of an acclimated BT pre-culture and incubated in a rotary shaker at 30 degree Celsius and 250 rounds per minute for 48 hours. At the end of the experiment, samples were taken aseptically for viable spore count and entomotoxicity bioassay. Procedure for viable spore count and bioassay are described below.

### Procedure for Evaluating BT Viable Spores Concentration

Yield of BT was evaluated in term of spores production. Viable spores may play a role in BT entomotoxicity and they are a the second major active ingredient of BT biopesticide formulation after insecticidal crystals. Viable spores count was performed by plate count technique according to APHA et al. (1989): (i) samples were serially diluted and previously heated at 70 degree Celsius during 15 minutes in heating bath; (ii) after these steps, samples were plated on tryptic soy agar and incubated at 30 degree Celsius during 16 hours in a incubator. Counts are reported as colony forming unit (CFU) per ml. The standard deviation for the method was estimated to approximately 8%.

### Procedure for Evaluating BT Entomotoxicity by Bioassay

Yield of BT was evaluated in term of insecticidal activity (BT entomotoxicity) against harmful insects. Entomotoxicity of BT subspecies kurstaki HD-1 was estimated by bioassay against third instar larvae of western spruce budworm (Choristoreuna occidentalis, Lepidoptera: Tortricidae) according to the diet incorporation method (Dulmage et al., 1971). Commercial preparation 76B Foray™ from Abbott Laboratories (Chicago, United States) was used as a standard. Larva of western spruce budworm were provided by the Canadian forest service of Natural Resources Canada (Ontario, Canada). If provided larva were in diapause, first or second instar, they were raised on a sterile artificial diet for 1 to 7 days, depending on the development stage to obtain third instar larva. The artificial diet for spruce budworm was supplied by the Division des forets of Natural Resources Ministry of Quebec (Quebec, Canada). The composition of the diet provided is presented in Table 2 below.

TABLE 2
Diet composition for spruce budworm larvae breeding.
Quantity for one liter

| Ingredients | | Quantity |
|---|---|---|
| Agar | g | 16.7 |
| Distilled water | ml | 840 |
| Casein (without vitamin) | g | 35 |
| 4 M potassium hydroxide | ml | 5 |
| Alphacel | g | 5 |
| Salt mixed (Wesson) | g | 10 |
| Sucrose | g | 35 |
| Wheat germ | g | 45.7 |
| Chloride choline | g | 1 |
| Vitamin solution¹ | g | 10 |
| Ascorbic acid | g | 4 |
| Formalin (37% formaldehyde) | g | 0.5 |
| Methylparaben | g | 1.5 |
| Aureomycin powder | g | 5.6 |

¹100 ml contain 100 mg of niacin, 100 mg of calcium pentothenate, 50 mg of riboflavin, 25 mg of thiamin hydrochloride, 25 mg of pyrodoxin hydrochloride, 25 mg of folic acid, 2 mg of biotin and 0.2 mg of B-12 vitamin.

The samples and the standard were serially diluted in a saline solution (0.85% NaCl) and three last dilutions were used for the test. For each dilution, 1 mL was deposed into 20 mL of sterile artificial diet for east spruce budworm containing 1.5% of sterile agar (Difco). Rapidly after properly mixing, 1 mL of mixture was deposited into 15×45 mm glass vials (VWR Canlab, Canada) with a perforated plastic cap. Vials were previously sterilized by autoclave (121° C., 15 min.) and caps under UV lights. Groups of 20 vials were used for each dilution. One larvae was delicately (and aseptically) transferred to each tube with a fine brush. Vials were then placed at room temperature under a light source (e.g. lamp with a 60 W bulb). Percentage mortality was evaluated after 7 days. Entomotoxicity values were calculated by comparing percentage mortality caused by diluted sample with percentage mortality of standard FORAY 76B™ (Abbott Laboratories, Chicago, US) at same dilution. Values of entomotoxicity are reported herein as international units per microliter (IU/μL). The standard deviation of the method was estimated to 7%. To determine whether waste water sludge affects the viability of larva, a group of 50 vials was used. The preparation was the same except that 2.5 mL of a serially diluted sludge sample was deposited into 50 mL of artificial diet before it was deposited in each vial. A group of 50 vials was used for the blank to test quality of artificial diet without larvae. The preparation was the same except that 2.5 mL of a saline solution (0.85% NaCl) was deposited into 50 mL of artificial diet before it was deposited in each vial. If the mortality in the control or blank vials was higher than 10%, the bioassay was repeated.

### Composition of Waste Water Sludge Used as Raw Material for BT Production

Two types of waste water sludges were used as raw material for BT production: municipal mixed sludges and secondary sludges. The mixed sludges initially contained between 1% to 5% of suspended solids (SS) and secondary sludge between 0.05% to 4%. The SS concentration was increased prior to applying the method of the present invention by settling and/or concentration using centrifugation (8000 revolution per minutes or 7650 g, 10 minutes, 4 degree Celsius) in a laboratory centrifuge. If necessary, SS may be adjusted by dilution with sludge supernatant obtained after centrifugation. SS concentration is desirably optimally adjusted for optimal BT production using waste water sludge as raw material. Also, adjusting SS concentration is a way to minimize wastewater sludge composition variability. Typical composition of mixed and secondary sludges used for BT production is defined in Table 3 below. Values of parameters are based on dry sludge (mg/kg dry sludge).

TABLE 3
Typical composition of municipal mixed and secondary waste water
sludge in mg/kg

| | Mixed | | Secondary | |
|---|---|---|---|---|
| Characteristics | Mean | Deviation | Mean | Deviation |
| Total carbon (mg C/kg) | 376000 | 8000 | 380000 | 40000 |
| Total nitrogen (mg N/kg) | 34000 | 9000 | 60000 | 10000 |
| Ratio C:N | 12 | 2.0 | 6.5 | 0.9 |
| Total organic carbon | 60000 | 20000 | 80000 | 30000 |
| (mg C/kg) | | | | |
| N—NH₄⁺ (mg N/kg) | 5000 | 2000 | 12000 | 11000 |
| N-organic (mg N/kg) | 28000 | 10000 | 50000 | 10000 |
| Al (mg/kg) | 15000 | 14000 | 20000 | 10000 |
| Ca (mg/kg) | 20000 | 1000 | 16000 | 7000 |
| Cd (mg/kg) | 1.1 | 0.6 | 1.4 | 0.9 |
| Cr (mg/kg) | 50 | 40 | 60 | 40 |
| Cu (mg/kg) | 440 | 90 | 200 | 100 |
| Fe (mg/kg) | 8000 | 7000 | 12000 | 7000 |
| K (mg/kg) | 20000 | 10000 | 6000 | 3000 |
| Mg (mg/kg) | 8000 | 4000 | 3000 | 2000 |
| Mn (mg/kg) | 300 | 100 | 150 | 60 |
| Mo (mg/kg) | 10 | 2 | 5 | 2 |
| Na (mg/kg)^{c} | 70000 | 60000 | 30000 | 10000 |
| Ni (mg/kg) | 20 | 8 | 14 | 9 |
| Pₜ (mg/kg) | 12000 | 1000 | 10000 | 4000 |
| Pb (mg/kg) | 30 | 20 | 30 | 10 |
| S (mg/kg) | 6000 | 2000 | 8000 | 7000 |
| Zn (mg/kg) | 1500 | 500 | 600 | 400 |

### EXAMPLE 2

### Thermal Alkaline Hydrolysis

The pH of 100 mL of mixed and secondary municipal waste water sludges was adjusted at 8.0±0.1 to 12.0±0.1 with a sodium hydroxyde solution. The sludges were then heated in a micro-wave digester Multiwave-microwave sample preparation system™ (Perkin Elmer & Paar Physica, US). The determined optimal range of temperature was 120 degree Celsius to 160 degree Celsius (shown in Table 4), but it is believed that a temperature of at least 180 degree Celsius could be used without deleteriously affecting the sludge properties. It is believed that compounds refractory to microbial growth and metabolite production may progressively be generated in the sludge beyond that temperature. Usually, a temperature of 120 degree Celsius is reached after heating for about 10 minutes. It is believed that heating more than 60 minutes at 180 degree Celsius and more than 120 minutes at 120 degree Celsius may deteriorate the sludge.

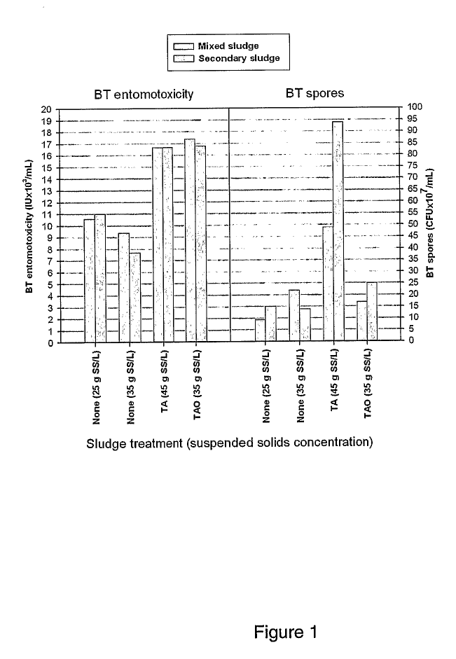

After this treatment, the sludge pH was adjusted aseptically to 7.0±0.2 with a H₂SO₄ solution for further microbial culture, before introducing BT. It should be noted that steam injection hydrolysis could also be used instead of the microwave hydrolysis. For small scale hydrolysis, a micro-wave digester is used to make thermal-alkaline hydrolysis. For high scale hydrolysis, steam injection hydrolysis can desirably be used. A possible procedure for steam injection hydrolysis consist in the use of a 10 L (or more) mechanical steam vessel stainless steel 316L with pure steam injection facility and controlled agitation (also referred to as a “hydrolyser”). Before hydrolysis, SS concentration is adjusted by taking into account dilution by steam. Such treatment also acts as a sterilization step. If treated sludge is not transferred aseptically to the shake flask or bioreactor, sterility may be lost. If sterility is lost, a further sterilization step (sterilization step at 121 degree Celsius during 30 minutes after adjusting pH to 7±0.2 with sulfuric acid solution or sodium hydroxide solution) may then be performed although without such step no deleterious effect was observed. See also FIG. 1 presenting the results with optimal parameters.

### EXAMPLE 3

### Thermal-Alkaline Hydrolysis Following by Partial Oxidation

Thermal-alkaline hydrolysis following by partial oxidation of mixed and secondary waste water sludges was performed in two steps. A thermal-alkaline hydrolysis was first performed as described in Example 2. An oxidative pre-treatment was then performed wherein the pH was adjusted with a sulfuric acid solution at a value of 3.0±0.1 (the optimal range is of about 2 to about 4) and 0.01 mL of hydrogen peroxide solution (30% v/v, Fisher) per gram of sludge SS (the optimal range is of about 0.01 to about 0.03 mL of hydrogen peroxide solution or about 3.19E-07 to about 9.58E-07 kg H₂O₂ per gram of SS) was added aseptically. The sludge was then placed in a heating rotary shaker bath at 70 degree Celsius (the optimal range of temperature is between about 25 and about 90 degree Celsius) in order to increase solubilization and at 60 rounds per minute (the optimal range is of about 30 to about 350 rounds per minute) for 2 hours (the optimal range is of about 1.5 to about 4 hours). The shaking, acidic conditions and high temperature favors the oxidation reaction and improve nutrient bioavailability by influencing conformation of extracellular polymers such as proteins in sludge.

Table 4 below presents the thermal-alkaline hydrolysis parameters that were used. The sludge pH was then adjusted aseptically to 7.0±0.2 with a sulfuric acid solution for further microbial culture, before introducing BT. Ranges have been established by a central composite design (CCD) using 4 independent variables. CCD has been defined with optimal conditions found to be: 35 g SS/L, pH 10, 140 degree Celsius, 30 minutes. Each point of CCD represents one shake flask experiment (K=extremity point, S=star point, C=central point). Seven replicates are done at the central point to confirm reproducibility. See also FIG. 1 presenting the results with optimal parameters.

TABLE 4
RANGE OF EACH PARAMETERS TESTED FOR
THERMAL-ALKALINE HYDROLYSIS IN EXAMPLE 2
AND FOR THERMAL-ALKALINE HYDROLYSIS
STEP IN EXAMPLE 3.
CCD of Examples 2 and 3

| Point in CCD | SS (g/L) | pH | Temperature (Celsius) | Length (h) |
|---|---|---|---|---|
| K1 | 30 | 9 | 130 | 20 |
| K2 | 30 | 9 | 150 | 20 |
| K3 | 30 | 11 | 130 | 20 |
| K4 | 30 | 11 | 150 | 20 |
| K5 | 30 | 9 | 130 | 40 |
| K6 | 30 | 9 | 150 | 40 |
| K7 | 30 | 11 | 130 | 40 |
| K8 | 30 | 11 | 150 | 40 |
| K9 | 40 | 9 | 130 | 20 |
| K10 | 40 | 9 | 150 | 20 |
| K11 | 40 | 11 | 130 | 20 |
| K12 | 40 | 11 | 150 | 20 |
| K13 | 40 | 9 | 130 | 40 |
| K14 | 40 | 9 | 150 | 40 |
| K15 | 40 | 11 | 130 | 40 |
| K16 | 40 | 11 | 150 | 40 |
| S1 | 25 | 10 | 140 | 30 |
| S2 | 45 | 10 | 140 | 30 |
| S3 | 35 | 8 | 140 | 30 |
| S4 | 35 | 12 | 140 | 30 |
| S5 | 35 | 10 | 120 | 30 |
| S6 | 35 | 10 | 160 | 30 |
| S7 | 35 | 10 | 140 | 10 |
| S8 | 35 | 10 | 140 | 50 |
| C1 | 35 | 10 | 140 | 30 |
| C2 | 35 | 10 | 140 | 30 |
| C3 | 35 | 10 | 140 | 30 |
| C4 | 35 | 10 | 140 | 30 |
| C5 | 35 | 10 | 140 | 30 |
| C6 | 35 | 10 | 140 | 30 |
| C7 | 35 | 10 | 140 | 30 |

TABLE 5
INDIVIDUAL RESULTS FOR SLUDGES TREATED ACCORDING TO
PARAMETERS PRESENTED IN TABLE 4

| | Results Example 2 | | | | Results Example 3 | | | |
|---|---|---|---|---|---|---|---|---|
| | Mixed sludge | | Sec. sludge | | Mixed sludge | | Sec. Sludge | |
| | Spores | Entomo. | Spores | Entomo. | Spores | Entomo. | Spores | Entomo. |
| CCD | (CFU × | (UI × 10³/ | (CFU × | (UI × 10³/ | (CFU × | (UI × 10³/ | (CFU × | (UI × 10³/ |
| points | 10⁷/ml)¹ | μl)¹ | 10⁷/ml)¹ | μl)¹ | 10⁷/ml)¹ | μl)¹ | 10⁷/ml)¹ | μl)¹ |
| K1 | 39 | 13.5 | 54 | 15.1 | 30 | 10.9 | 10 | 12.6 |
| K2 | 42 | 13.8 | 66 | 13.7 | 39 | 11.0 | 25 | 11.9 |
| K3 | 33 | 14.2 | 77 | 15.2 | 39 | 13.1 | 24 | 15.8 |
| K4 | 35 | 15.5 | 65 | 14.4 | 28 | 15.0 | 19 | 14.7 |
| K5 | 41 | 11.8 | 129 | 14.5 | 27 | 10.9 | 10 | 13.5 |
| K6 | 41 | 12.4 | 94 | 16.7 | 20 | 13.0 | 18 | 16.6 |
| K7 | 40 | 13.5 | 106 | 14.2 | 27 | 12.6 | 17 | 15.9 |
| K8 | 38 | 14.1 | 142 | 14.1 | 17 | 10.8 | 15 | 14.1 |
| K9 | 36 | 12.8 | 30 | 16.1 | 35 | 14.2 | 64 | 12.3 |
| K10 | 52 | 11.7 | 51 | 11.4 | 11 | 13.4 | 20 | 13.4 |
| K11 | 40 | 13.7 | 19 | 12.0 | 49 | 14.3 | 44 | 12.6 |
| K12 | 42 | 13.8 | 28 | 15.1 | 26 | 15.8 | 25 | 16.8 |
| K13 | 47 | 16.4 | 36 | 13.7 | 37 | 15.2 | 15 | 14.3 |
| K14 | 50 | 14.3 | 33 | 14.4 | 47 | 16.8 | 36 | 13.6 |
| K15 | 50 | 16.2 | 48 | 13.7 | 17 | 15.1 | 25 | 11.7 |
| K16 | 41 | 15.5 | 11 | 13.8 | 14 | 14.6 | 41 | 8.7 |
| S1 | 58 | 13.3 | 116 | 13.1 | 92 | 14.2 | 22 | 14.6 |
| S2 | 49 | 16.7 | 58 | 10.9 | 20 | 12.0 | 31 | 16.0 |
| S3 | 46 | 11.0 | 49 | 10.4 | 17 | 17.5 | 29 | 11.6 |
| S4 | 51 | 15.4 | 80 | 9.1 | 26 | 10.3 | 19 | 13.6 |
| S5 | 55 | 14.5 | 113 | 13.1 | 82 | 11.9 | 32 | 10.8 |
| S6 | 52 | 14.4 | 42 | 13.8 | 9 | 11.1 | 25 | 14.5 |
| S7 | 55 | 12.0 | 24 | 9.6 | 12 | 12.9 | 17 | 11.5 |
| S8 | 53 | 13.4 | 47 | 11.4 | 14 | 11.4 | 38 | 15.4 |
| C1 | 51 | 14.8 | 55 | 14.0 | 24 | 12.4 | 36 | 14.2 |
| C2 | 47 | 14.7 | 48 | 12.1 | 11 | 10.9 | 46 | 11.8 |
| C3 | 58 | 15.2 | 65 | 12.3 | 16 | 13.3 | 28 | 13.4 |
| C4 | 60 | 13.7 | 37 | 10.8 | 10 | 14.7 | 26 | 16.4 |
| C5 | 50 | 14.1 | 25 | 9.9 | 14 | 11.8 | 19 | 13.3 |
| C6 | 60 | 15.1 | 54 | 12.8 | 15 | 13.1 | 14 | 15.3 |
| C7 | 51 | 15.5 | 30 | 11.7 | 23 | 12.4 | 22 | 14.3 |

¹CFU = Colony forming unit according to plate count technique described in APHA & al. (1989). IU = International units according to BT entomotoxicity test described in Dulmage & al. (1971). Standard deviations for viable spores yield and entomotoxicity were 8.0% and 7.0% respectively.

### EXAMPLE 4

### BT Spore Production and Entomotoxicity after Treatments

The effect on BT spore production and entomotoxicity of the treatments described in Examples 2 and 3 above is shown in Tables 5 and 6. Increasing SS of sludge from 25 g/L to 35 g/L was shown to decrease entomotoxicity of BT produced in raw mixed or secondary sludge. Examples 2 and 3 show that thermal-alkaline hydrolysis, and thermal-alkaline hydrolysis followed by partial oxidation of waste water sludge, allow the use of higher SS concentration in sludges for BT production. Higher entomotoxicities and spore concentrations have been obtained in sludge containing high SS concentration when sludges were pre-treated with thermal-alkaline hydrolysis, or thermal-alkaline hydrolysis following by partial oxidation.

By comparison with BT produced in raw mixed sludge, at 25 g SS/L, thermal-alkaline hydrolysis and thermal-oxidative pre-treatment increased entomotoxicity by 58% and 64%, respectively, and spore concentration by 4.2 and 0.8 fold, respectively. By comparison with BT produced in raw secondary sludge, at a concentration of 25 g SS/L, thermal-alkaline hydrolysis, and thermal-alkaline hydrolysis following by partial oxidation, increase entomotoxicity by 52% and 53%, respectively, and spores concentration by 5.3 and 6.7 fold respectively.

TABLE 6
ENTOMOTOXICITY VALUES OF BT AFTER 48 H IN
SHAKE FLASK MICROBIAL CULTURE WITH PRE-
TREATED OR RAW WASTEWATER SLUDGE
(MIXED OR SECONDARY) AND CORRESPONDING
SS CONTENT AND SPORES CONCENTRATIONS

| | | BT entomotoxicity | BT spores |
|---|---|---|---|
| Sludge | Pre-treatment | (IU × 10³/μL)^{2,3} | (CFU × 10⁷/mL)² |
| Mixed | None (25 g SS/L)¹ | 10.6 | 9.5 |
| | None (35 g SS/L)¹ | 9.4 | 22 |
| | Thermal-alkaline | 16.7 | 49 |
| | (45 g SS/L) | | |
| | Thermal-alkaline | 17.4 | 17 |
| | hydrolysis following | | |
| | by partial oxidation | | |
| | (35 g SS/L) | | |
| Secondary | None (25 g SS/L)¹ | 11.0 | 15 |
| | None (35 g SS/L)¹ | 7.7 | 14 |
| | Thermal-alkaline | 16.7 | 94 |
| | (30 g SS/L) | | |
| | Thermal-alkaline | 16.8 | 25 |
| | hydrolysis following | | |
| | by partial oxidation | | |
| | (40 g SS/L) | | |

¹Raw sludge: BT production in raw sludge containing 25 or 35 g SS/L. Viable spores and entomotoxicity yield are the mean of three replicates.
²CFU = Colony forming unit according to plate count technique. IU = International units according to BT entomotoxicity test. Standard deviations for viable spores yield and entomotoxicity were 8.0% and 7.0% respectively.
³Maximal entomotoxicity values achieved with CCD for BT production in pre-treated sludge.

### EXAMPLE 5

### Determination of Correlation Between Cell Growth and Entomotoxicity

Table 7 below shows that there is not correlation between the ability of a media to increase BT cell growth and its ability to increase BT entomotoxicity.

TABLE 7

| | Viable cells | Entomotoxicity |
|---|---|---|
| Microbial culture substrat | (CFU × 10⁷/ml)*** | (IU/μl)*** |
| Soya* | 63.8 | 6926 |
| Raw mixed waste water sludges** | 39.0 | 10819 |

*The << soya >> medium is a prior art synthetic medium for producing BT kurstaki. It contains glucose, starch, soya flour and mineral salts.
**Mixed sludges used contained 25 g of SS per liter. Their composition is as described herein.
***Experiments in duplicata in Erlenmeyers. Microbial culture conditions are the same as those described above. The standard deviation of the procedure for counting cells is of 8% and that of the bioassays to evaluate entomotoxicity is of 7%.

### EXAMPLE 6

### Trichoderma Production

### Sludge Pre-Treatments (Hydrolysis Step)

The conditions used to hydrolyse the sludges were identical to those described in Example 2 for growing Bacillus thuringiensis (parameters of the central point in CCD namely the determined optimal conditions for BT.

### Growing Trichoderma in Sludge

### Starter Culture and Inoculum

The starter culture consisted of ≈½″×½″ scraped piece of 32-36 h old mycelial mat of a commercial strain of Trichoderma viride, cultured on PDA plate at 28° C. and ≈35% relative humidity. In order to prepare inoculation for the process medium (waste), a single piece of above mentioned starter culture was inoculated into 500 ml Erlenmeyer flask containing 150 ml of sterile tryptic soya broth (TSB, Difco). The sterilization of the TSB medium was carried out at 121° C. for 15 minutes in a wet autoclave (Sanyo Laboautoclave—Sanyo™, Japan) after adjusting the medium pH to 6.1±0.1 with 2N H₂SO₄, or 2N NaOH solution. The Erlenmeyer flasks were incubated in duplicate in a rotary shaker (Model-G4, New Brunswick Scientific) at 28° C. and 250±10 rpm for 48 h. It is expected that a pH between about 5.6 and 6.4 will be appropriate for Trichoderma culture.

### Microbial Culture Protocol

The incubation of Trichoderma fungi in sludge was carried out in a manner similar as described above for the inoculum in TSB except that the sterilization of the sludge was carried out at 121° C. for 30 minutes.

### Bioassay Against Insect

The Trichoderma viride culture, grown in raw sludge (NH) and in thermal alkaline treated sludge (TAH) were subjected to bioassays as described in Examples 2 and 3 above for Bacillus thuringiensis.

### Results

### Growth in Sludge (Spore/Conidia Production)

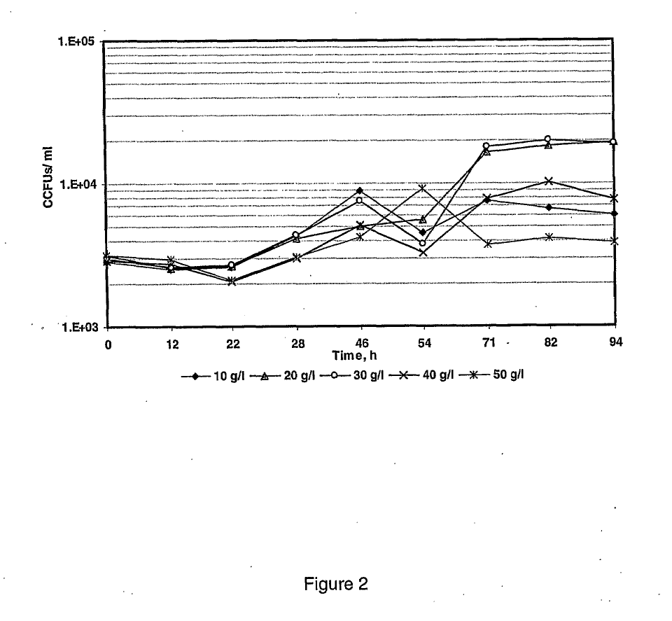

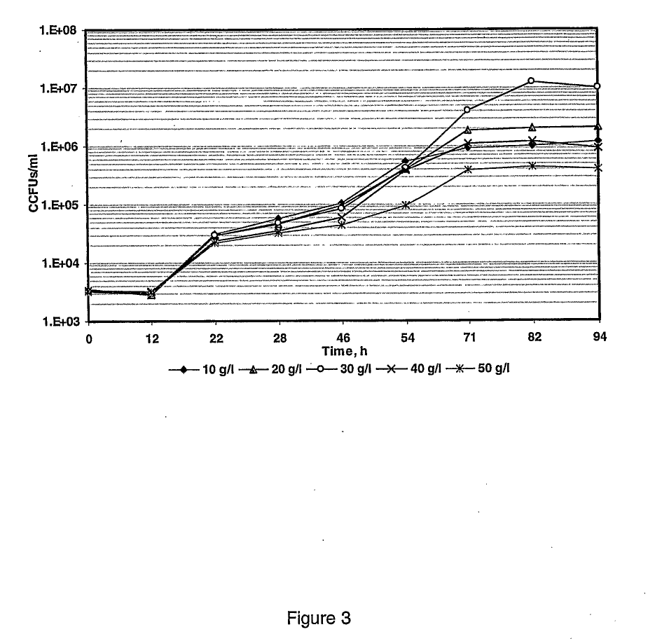

The conidial colony forming unit (i.e. viable conidia) production is presented in Table 8 below and in FIGS. 2 and 3.

TABLE 8
Maximum conidial spore production in sludge

| | | |
|---|---|---|
| | Maximum conidia* | % Increase in |
| Suspended | (CFU/ml) | TAH as |

| solids (g/l) | NH | TAH | compared to NH |
|---|---|---|---|
| 10 | 7500 | 1200000 | 15900 |
| 20 | 19100 | 2100000 | 10895 |
| 30 | 19800 | 12200000 | 61516 |
| 40 | 10300 | 12000000 | 116405 |
| 50 | 4100 | 430000 | 10388 |

*8% Standard deviation

TABLE 9
Conidia (spores) production of Trichoderma viride in pre-treated or raw
secondary wastewater sludge. Results are presented in CFU/ml
(conidial colony forming units per ml)

| Pre- | Culture time (h) | | | | |
|---|---|---|---|---|---|
| treatment* | 46 | 54 | 71 | 82 | 94 |
| None | 7.6 × 10³ | 4.8 × 10³ | 2.9 × 10⁴ | 3.1 × 10⁴ | 3.0 × 10⁴ |
| Thermal- | 8.5 × 10⁴ | 4.4 × 10⁵ | 4.0 × 10⁶ | 1.4 × 10⁷ | 1.0 × 10⁷ |
| alkaline | | | | | |

*SS was adjusted to 30 g SS/I. Thermal alkaline hydrolysis conditions: pH 10, 140° C., 30 min.

Thermal alkaline hydrolysis increased the CFU counts at all solid concentrations tested. At solids concentrations above 30 g/l, factors like O₂ transfer and osmotic pressure could adversely affect conidiation.

### Bioassay Against Insect

The entomotoxicity of Trichoderma grown in TSB was about 6578 IU/μl. The results of entomotoxicity are presented in Table 10, showing an entomotoxicity increase of between 30-36% in thermal alkaline treatment as compared to raw sludge at different suspended solid concentrations. The entomotoxicity increase in raw sludge and thermal alkaline treated sludges as compared to TSB was between 6-129% at different solids concentrations.

TABLE 11
Entomotoxicity (Tx) in raw sludge and thermal alkaline treatment at
different solids concentration

| Suspended solids | Tx (IU/μl) | | |
|---|---|---|---|
| (gl⁻¹) | NH | TAH | Percent increase |
| 10 | 6971 | 9042 | 29.7 |
| 20 | 9850 | 12945 | 31.4 |
| 30 | 11051 | 15036 | 36.1 |
| 40 | 7289 | 9564 | 31.2 |

The methods of the present invention for growing Trichoderma sp., achieved a high spore production. It achieved approximately a 10 to 1000-fold increase in conidia production of Trichoderma viride for a culture time of 46 to 94 h.

Although the present invention has been described hereinabove by way of preferred embodiments thereof, it can be modified, without departing from the spirit and nature of the subject invention as defined in the appended claims.

### REFERENCES

Babu, R. M., A. Sajeenaa, K. Seetharamana, P. Vidhyasekarana, P. Rangasamy, M. S. Prakash, A. S. Rajab and K. R. Biji (2003). Advances in bioherbicides development—an overview. Crop Protection, 22: 253-260. Ben Rebah, F., R. D. Tyagi & D. Prévost (2001). Acid and alkaline treatment for enhancing the growth of rhizobia in sludge. Can. J. Microbiol., 47 (6): 467-474.
Benhamou, N. and Brodeur, J. 2000. Evidence for Antibiosis and Induced Host Defense Reactions in the Interaction Between Verticillium lecanii and Penicillium digitatum, the Casual Agent for Green Mold. Biochemistry and Cell Biology, 90(9): 932-943.
Benhamou, N., Rey, P., Picard, K., Tirilly, Y. 1999. Ultrastructural and cytochemical aspects of the interaction between the mycoparasite, Pythium oligandrum, and soilborne pathogens. Phytopathology, 89: 506-517.
Bonnarme, P., A. Djian, A. Latrasse, G. Feron, C. Ginies, A. Durand and J. L. Le Quéré (1997). Production of 6-pentyl-a-pyrone by Trichoderma sp. from vegetable oils. J. Biotechnol., 56: 143-150.
Calistru, C., McLean, M., Berjak, P. 1997a. In vitro studies on the potential for biological control of Aspergillus flavus and Fusarium moniliforme by Trichoderma species. A study of the production of extracellular metabolites by Trichoderma species. Mycopathologia, 137: 115-124.
Calistru, C., McLean, M., Berjak, P. 1997b. In vitro studies on the potential for biological control of Aspergillus flavus and Fusarium moniliforme by Trichoderma species 1. Macroscopical and microscopical observations of fungal interactions. Mycopathologia, 139: 115-121
Celar, F. 2003. Competition for ammonium and nitrate forms of nitrogen between some phytopathogenic and antagonistic soil fungi. Biological Control, 28: 19-24.
Chet, I. 1993. Biotechnology in Plant Disease Control. John Wiley & Sons, New York.
Copping, L. G. and J. J. Menn (2000). Biopesticides: a review of their action, application and efficacy. Pest. Manag. Sci., 56: 651-676.
Dulmage, H. T., O. P. Boening, C. S. Rehnborg et G. D. Hansen (1971). A proposed standardized bioassay for formulations of Bacillus thuringiensis based on the international unit. J. Inv. Pathol., 18: 240-245.
Ejechia, B. O. 1997. Biological control of wood decay in an open tropical environment with Penicillium spp. and Trichoderma viride. International Biodeterioration and Biodegradation, 39: 295-299.
Esposito, E., da Silva, M. 1998. Systematics and Environmental Application of the Genus Trichoderma, Critical Reviews in Microbiology, 24(2): 89-98.
Felse, P. A., Panda, T. 2000. Submerged culture production of chitinase by Trichoderma harzianum in stirred tank bioreactors—the influence of agitator speed. Biochemical Engineering Journal, 4: 115-120.
Feng, G. B., T. J. Poprawski and G. G. Khachatourians (1994). Production, formulation and application of the entomopathogenic fungus Beauveria bassiana for insect control: current status. Biocontrol Sci. Technol., 4: 3-34.
Harman, G. E., Haynes, C. K., Lorito, M., Broadway, R. M., di Pietro, A., Peterbauer, C., Tronsmo, A. 1993. Chitinolytic enzymes of Trichoderma harzianum: purification of chitobiosidase and endochitinase. Phytopathology, 83: 313-318.
Harmann, G. E. and Bjorjmann, T. 1998. Potential and existing uses of Trichoderma and Gliocladium for plant disease control and plant growth enhancement. In Trichoderma and Gliocladium Vol. 2. (Harmann, G. E. and Kubicek, C. K., eds), pp 229-265, Taylor and Francis Ltd., London.
Howard, R. L., E. Abotsi, E. L. Jansen van Rensburg and S. Howard (2003). Lignocellulose biotechnology: issues of bioconversion and enzyme production. African Journal of Biotechnology, 2 (12): 602-619. Hutchinson, C. M. (1999). Trichoderma virens—Inoculated Composted Chicken Manure for Biological Weed Control. Biological Control, 16: 217-222. Jones, R. W., W. T. Lanini and J. G. Hancock (1988). Plant growth response to the phytotoxin viridiol produced by the fungus Gliocladium virens. Weed Sci., 36: 683-687. Lachhab, K., R. D. Tyagi & J. R. Valéro (2001). Production of Bacillus thuringiensis biopesticides using wastewater sludge as a raw material: effect of inoculum and sludge solids concentration. Process Biochem., 37: 197-208.
Lin, A., Lee, T. M. and Rern, J. C. 1994. Tricholin, a new antifungal agent from Trichoderma viride, and its action in biological control of Rhizoctonia solani. Journal of Antibiotics, 47: 799-805.
Lisansky, S., Quinlan, R. J. & G. Tassoni (1993). The Bacillus thuringiensis production handbook. CPL Scientific Press, Newbury, UK, 124 p.
Lisboa De Marco, J., Valadares-Inglis, M. C., Felix, C. R. 2003. Production of hydrolytic enzymes by Trichoderma isolates with antagonistic activity against Crinipellis perniciosa, the causal agent of witches' broom of cocoa. Brazilian Journal of Microbiology, 34: 33-38.
Ortiz, A. and S. Orduz (2000). In vitro evaluation of Trichoderma and Gliocladium antagonism against the symbiotic fungus of the leaf-cutting ant Atta cephalotes. Mycopathologia, 150: 53-60. Papavizas, G. C. 1985. Trichoderma and Gliocladium: biology, ecology and potential for biocontrol. Annual Review of Phytopathology, 23: 23-54.
Pelczar Jr., M. J., Chan, E. C. S., Kreig N. R. Microbiology, 5^{th} ed., Tata McGraw-Hill, Inc. New York, 1993, ISBN 0-07-049234-4.
Samuels, G. J., Chaverri, P., Farr, D. F., McCray, E. B. Trichoderma Online, Systematic Botany & Mycology Laboratory, ARS, USDA. Retrieved Jun. 15, 2004, from http://nt.ars-grin.gov/taxadescriptions/keys/TrichodermaIndex.cfm.
Saxena, R. K., Gupta, R., Kuhad, R. C., Khurana, N. 1993. Light independent conidiation in Trichoderma spp., a novel approach to microcycle conidiation. World Journal of Microbiology, 9: 353-356.
Schnepf, E., N. Crickmore, J. Van Rie, D. Lereclus, J. Baum, J. Feitelson, D. R. Zeigler et D. H. Dean (1998). Bacillus thuringiensis and its pesticidal crystal proteins. Microbiol. Mol. Biol. Rev., 62 (3): 775-806.
St-Germain, G. and Summerbell, R. (eds) 1996. Identifying Filamentous Fungi—A Clinical Laboratory Handbook, 1st ed. Star Publishing Company, Belmont, Calif.
Steinmetz, J. and F. Schönbeck (1994). Conifer bark as growth medium and carrier for Trichoderma harzianum and Gliocladium roseum to control Pythium ultimum on pea. J. Plant Diseases Prot., 101: 200-210. Tirado-Montiel, M. T. L. (1997). Utilisation des boues des usines de traitement comme moyen alternatif pour la production de l'insecticide microbien Bacillus thuringiensis. Ph. D. thesis, INRS-Eau, Université du Québec, Quebec, Canada.
Tirado-Montiel, M. T. L., R. D. Tyagi & J. R. Valéro (1998). Production of Bacillus thuringiensis using waste materials. In Martin, A. M. (ed.), Bioconversion of waste materials to industrial products, Blackie Academic Press & Professional, London, UK, p. 480-516.
Tirado-Montiel, M. T. L., R. D. Tyagi & J. R. Valero (2001). Wastewater treatment sludge as a raw material for the production of Bacillus thuringiensis based biopesticides. Wat. Res., 35 (16): 3807-3816.
Vidyarthi, A. S., R. D. Tyagi, J. R. Valéro & R. Y. Surampalli (2002). Studies on the production of B. thuringiensis using wastewater sludge as a raw material. Wat. Res., 36: 4850-4860.

1. A media for growing a biopesticide producing microorganism comprising waste water sludge having undergone thermal alkaline hydrolysis performed by adjusting the pH of the wastewater sludge between about 8 and about 12 with an alkaline solution selected from the group consisting of NaOH, KOH, CaOH₂ and MgOH₂ at a temperature between about 120 and about 180 degree Celsius.

2. The media of claim 1, wherein said thermal alkaline hydrolysis is performed for at least about 10 minutes to about 50 minutes.

3. The media of claim 1, wherein the sludge was oxidized after the heating step.

4. The media of claim 3, wherein the step of oxidizing the sludge was performed by adjusting the pH with a sulfuric acid solution at about 1.5 to about 4 and adding 3.19E-07 to 9.58E-07 kg H₂O₂ per gram of SS.

5. The media of claim 3 or 4, wherein the sludge was placed in a heating bath up to 70 degree Celsius for about 1.5 to 4 hours after the oxidation step.

6. The media claim 1, wherein the sludge has been subjected, after thermal alkaline hydrolysis, to a step of adjusting the sludge's pH with an acid which does not have an inhibitory effect on biopesticide producing microorganism growth.

7. The media of claim 6, where said acid is H₂SO₄.

8. The media of claim 1, wherein said sludge has a concentration in solids between about 10 g SS/L and about 50 g SS/L.

9. The media of claim 1, wherein said biopesticide producing microorganism is a biopesticide producing bacteria.

10. The media of claim 1, wherein said biopesticide producing microorganism is a biopesticide producing Bacillus thuringiensis (BT).

11. The media of claim 10, wherein said biopesticide producing BT is selected from the group consisting of BT serovar israelensis; BT biovar tenebrionis; BT serovar japonensis; and BT serovar aizawai.

12. The media of claims 1, wherein said biopesticide producing microorganism is a biopesticide producing fungus.

13. The media of claim 1, wherein said biopesticide producing microorganism is a biopesticide producing Trichoderma spp.

14. A method for increasing the bioavailability of nutrients in waste water sludge for biopesticide producing microorganisms, comprising subjecting the sludge to a thermal alkaline pre-treatment comprising adjusting the sludge pH to between about 8 and about 12 at a temperature between about 120 and 180 degree Celsius for a time sufficient to increase the bioavailability of nutrients in said sludge.

15. A method of increasing the pesticidal activity of a biopesticide producing microorganism, comprising
growing a biopesticide producing microorganism in a culture media as recited in claim 1.

16. The method of claim 15, wherein said biopesticide producing microorganism is a biopesticide producing bacteria.

17. The method of claim 16, wherein said biopesticide producing microorganism is a biopesticide producing Bacillus thuringiensis (BT).

18. The method of claim 17, wherein said biopesticide producing BT is selected from the group consisting of BT serovar israelensis; BT biovar tenebrionis; BT serovar japonensis; and BT serovar aizawai.

19. The method of claim 15, wherein said biopesticide producing microorganism is a biopesticide producing fungus.

20. The method as of claim 16, wherein said biopesticide producing microorganism is a biopesticide producing Trichoderma spp.

21. A method of increasing the pesticidal activity of a biopesticide producing microorganism, comprising
(a) subjecting waste water sludge to a thermal alkaline pre-treatment comprising adjusting the sludge pH to between about 8 and between about 12 at a temperature between about 120 and 180 degree Celsius for a time sufficient to increase the bioavailability of nutrients in said sludge;
(b) adjusting the pH of the sludge to provide appropriate growth conditions for the biopesticide producing microorganism; and
(c) growing the biopesticide producing microorganism in the sludge of step (b).

22. The method of claim 21, wherein said thermal alkaline hydrolysis is performed for at least about 10 minutes.

23. The method of claim 21, further comprising the step of oxidizing the sludge after step (a).

24. The method of claim 23, wherein the step of oxidizing the sludge is performed by adjusting the pH with a sulfuric acid solution at about 1.5 to about 4 and adding 3.19E-07 to 9.58E-07 kg of H₂O₂ per gram of SS.

25. The method of claim 24, further comprising after the oxidation step, the step of placing the sludge in a heating bath at about 25 to 70 degree Celsius for about 1.5 to 4 hours.

26. The method of claim 21, wherein said sludge has a concentration in solids between about 10 g SS/L and about 50 g SS/L prior step (a).

27. The method of claim 21, wherein said biopesticide producing microorganism is a biopesticide producing bacteria.

28. The method of claim 21, wherein said biopesticide producing microorganism is a biopesticide producing Bacillus thuringiensis (BT).

29. The method of claim 28, wherein said biopesticide producing BT is selected from the group consisting of BT serovar israelensis; BT biovar tenebrionis; BT serovar japonensis; and BT serovar aizawai.

30. The method of claim 27, wherein the pH to which the sludge is adjusted at step (b) is 7.0±0.2.

31. The method of claim 21, wherein said biopesticide producing microorganism is a biopesticide producing fungus.

32. The method of claim 21, wherein said biopesticide producing microorganism is a biopesticide producing Trichoderma spp.

33. The method of claim 31, wherein the pH to which the sludge is adjusted at step (b) is 6.1±0.1.

34. The method of claim 30, wherein the pH is adjusted with H₂SO₄.

35. The method of claim 15, wherein said pesticidal activity is entomotoxicity.

36. A biologically pure biopesticide producing microorganism grown in a culture media as recited in claim 1.

37. A biologically pure biopesticide producing microorganism produced by the method of claim 14.
